(19) United States
(12) Patent Application Publication
(10) Pub. No.: US 2017/0058352 A1
(43) Pub. Date: Mar. 2, 2017

(54) DIAGNOSTIC USE OF VIRUS-ENCODED MICRO RNA HSV1-MIR-H18 AND HSV2-MIR-H9-5P ON PROSTATE CANCER

(71) Applicant: CHUNGBUK NATIONAL UNIVERSITY INDUSTRY ACADEMIC COOPERATION FOUNDATION, Chungcheongbuk-do (KR)

(72) Inventors: Wun-Jae KIM, Seoul (KR); Seok Joong YUN, Chungcheongbuk-do (KR); Pil Du JEONG, Chungcheongbuk-do (KR); Jung Min KIM, Seoul (KR)

(21) Appl. No.: 15/112,641

(22) PCT Filed: Mar. 17, 2015
(86) PCT No.: PCT/KR2015/002562

§ 371 Date: Jul. 19, 2016

(30) Foreign Application Priority Data
Mar. 17, 2014 (KR) 10-2014-0031277

(51) Int. Cl. C12Q 1/68; C12Q 1/70

(52) U.S. Cl. CPC C12Q 1/6886; C12Q 1/705; C12Q 2600/158; C12Q 2600/178

(57) ABSTRACT

The present invention provides a novel use of hsvI-miR-H18 or hsv2-miR-H9-5p, which is microRNA, for the diagnosis of prostate cancer. According to hsvI-miR-H18 or hsv2-miR-H9-5p of the present invention, it is possible to accurately diagnose even a subject in the PSA gray zone in which accurate diagnosis though PSA is difficult. The present invention can be applied to various biological samples, and exerts accurate diagnostic ability even when a supernatant of the solution excluding, particularly, cell-derived materials is used, and thus a diagnostic procedure is simple and convenient. According to the present invention, the prostate cancer can be accurately and promptly diagnosed at low costs.

### FIELD

The present invention relates to a diagnostic use of virus-encoded microRNAs hsv1-miR-H18 and hsv2-miR-H9-5p on prostate cancer.

### BACKGROUND

Prostate cancer (CaP) is one of the most prevalent malignant diseases among men in Western countries, and the incidence thereof has also increased in Asian men. The early diagnosis of prostate cancer is possible by determining prostatic-specific antigen (PSA) levels, but PSA is not specific to only prostate cancer. The detection rate of prostate cancer through transrectal prostate biopsy reaches about 30%, but approximately 70% of those patients underwent this unnecessary and unpleasant procedure. Therefore, novel biomarkers for the detection of prostate cancer with still higher accuracy and convenience is required.

MicroRNAs (miRNAs), which are non-protein-encoding RNA regulators, have, on average, a 22-nucleotide length and are implicated in numerous biological and developmental processes. Approximately 50% of human miRNAs are encoded in genomic regions and are frequently altered in various types of cancers. It has been suggested that miRNAs may be used as potent biomarkers since stable miRNAs have been detected in biological samples, such as serum, plasma, and urine. In reference to prostate cancer, several study results have been reported that putative miRNAs in body fluids are used as diagnostic and prognostic markers. However, the complex diagnostic methodology and low reliability (due to relatively small numbers of test target patients) have low clinical applications of these studies.

Throughout the entire specification, many papers and patent documents are referenced and their citations are represented. The disclosure of cited papers and patent documents is entirely incorporated by reference into the present specification, and the level of the technical field within which the present invention falls and details of the present invention are explained more clearly.

### DETAILED DESCRIPTION

### Technical Problem

The present inventors have carried out in-depth studies to discover biomarker materials capable of accurately diagnosing prostate cancer in early stages in biological samples. As a result, the present inventors have experimentally demonstrated that virus-derived microRNA hsv1-miR-H18 or hsv2-miR-H9-5p can be favorably used for the diagnosis of prostate cancer since it is expressed at a significantly high level in biological samples from prostate cancer patients, and thus have completed the present invention.

An aspect of the present invention is to provide a composition for the diagnosis of prostate cancer.

Another aspect of the present invention is to provide a kit for the diagnosis of prostate cancer.

Still another aspect of the present invention is to provide a biomarker for the diagnosis of prostate cancer.

Still another aspect of the present invention is to provide a method for determining the expression level of microRNA, which is a biomarker for prostate cancer, to provide information necessary for the diagnosis of prostate cancer.

Still another aspect of the present invention is to provide a method for the diagnosis of prostate cancer, the method including a step of determining the expression level of microRNA.

Other purposes and advantages of the present disclosure will become more obvious with the following detailed description of the invention, claims, and drawings.

### Technical Solution

In accordance with an aspect of the present invention, there is provided a composition for the diagnosis of prostate cancer, containing an agent capable of detecting microRNA hsv1-miR-H18 or hsv2-miR-H9-5p.

The present invention provides a novel use of microRNA hsv1-miR-H18 or hsv2-miR-H9-5p for the diagnosis of prostate cancer.

Herein, hsv1-miR-H18 or hsv2-miR-H9-5p is a microRNA molecule known to be encoded by the virus. The nucleotide sequences of hsv1-miR-H18 and hsv2-miR-H9-5p of the present invention are represented by SEQ ID NO: 15 and SEQ ID NO: 16, respectively.

The present inventors have validated that hsv1-miR-H18 or hsv2-miR-H9-5p is expressed at a significantly high level in a prostate cancer patient, and experimentally demonstrated that the accurate diagnosis of prostate cancer can be achieved by determining the expression level of microRNA in a biological sample. As far as the present inventors know, it is first established that the virus-encoded microRNA hsv1-miR-H18 or hsv2-miR-H9-5p is correlated with prostate cancer.

Particularly, the microRNA marker of the present invention enables accurate diagnosis even on a patient within the prostate-specific antigen (PSA) gray zone (≦3 ng/ml and ≧10 ng/ml), in which accurate diagnosis is not attained by the conventional prostate cancer marker PSA array.

Herein, the agent capable of detecting microRNA RNA hsv1-miR-H18 or hsv2-miR-H9-5p refers to an agent that specifically binds to or reacts with microRNA to verify the presence or concentration of microRNA. Specific examples thereof include aptamers, nucleic acids, oligonucleotides, antibodies or fragments thereof, peptides, and peptide nucleic acids (PNAs), which can bind to microRNA hsv1-miR-H18 or hsv2-miR-H9-5p, but are not limited thereto.

According to a preferable embodiment of the present invention, the agent capable of detecting microRNA hsv1-miR-H18 or hsv2-miR-H9-5p is a primer or probe having a sequence that is complementary to the nucleotide sequence of hsv1-miR-H18 or hsv2-miR-H9-5p.

As used herein, the term “complementary” refers to having such complementarity to be selectively hybridizable with the nucleotide sequence of hsv1-miR-H18 or hsv2-miR-H9-5p under specific hybridization or annealing conditions. Therefore, the term “complementary” has a different meaning from being perfectly complementary. The primer or probe of the present invention may have one or more mismatch nucleotide sequences as long as it is selectively hybridizable with the nucleotide sequence of hsv1-miR-H18 or hsv2-miR-H9-5p.

As used herein, the term “probe” refers to a linear oligomer of natural or modified monomers or linkages, including deoxyribonucleotides and ribonucleotides, which are capable of specifically hybridizing with a target nucleotide sequence, and exists naturally or is artificially synthesized. The probe according to the present invention preferably has a single-stranded form. Preferably, the probe is an oligodeoxyribonucleotide. The probe of the present invention may include naturally occurring dNMPs (i.e., dAMP, dGMP, dCMP and dTMP), or nucleotide analogs or derivatives. The probe of the present invention may also include ribonucleotides. For example, the probe of the present invention may include: backbone-modified nucleotides, such as peptide nucleic acid (PNA) (M. Egholm et al., Nature, 365:566-568 (1993)), phosphorothioate DNA, phosphorodithioate DNA, phosphoramidate DNA, amide-linked DNA, MMI-linked DNA, 2′-O-methyl RNA, alpha-DNA and methylphosphonate DNA; sugar-modified nucleotides, such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA; and base-modified nucleotides, such as C-5 substituted pyrimidine (substituent including fluoro-, bromo-, chloro-, iodo-, methyl-, ethyl-, vinyl-, formyl-, ethynyl-, propynyl-, alkynyl-, thiazolyl-, imidazolyl-, and pyridyl-), 7-deazapurines with C-7 substituent (substituent including fluoro-, bromo-, chloro-, iodo-, methyl-, ethyl-, vinyl-, formyl-, alkynyl-, alkenyl-, thiazolyl-, and imidazolyl-, pyridyl-), inosine, and diaminopurine.

As used herein, the term “primer” refers to a single-strand oligonucleotide capable of functioning as an initial point of the template-directed DNA synthesis in an appropriate buffer under an appropriate condition (i. g., four different nucleoside triphosphates and polymerase) at a proper temperature. The appropriate length of the primer may vary according to various factors, for example, temperature, and a use of the primer, but the primer is, typically, 15- to 30-nucleotide in length. Short primer molecules generally require a lower temperature in order to form sufficiently stable hybrid complexes together with templates. The sequence of the primer does not necessarily need to be perfectly complementary to some sequences of the template, but the primer should have sufficient complementarity within the range where it can perform its inherent actions through the hybridization with the template. Accordingly, the primer of the invention does not necessarily need to have a sequence that is perfectly complementary to the nucleotide sequence of hsv1-miR-H18 or hsv2-miR-H9-5p, but should have sufficient complementarity within the range where the primer can act as a primer through the hybridization with this sequence.

According to a preferable embodiment of the present invention, the primer is used for a gene amplification reaction. As used herein, the term “amplification reaction” refers to a reaction of amplifying a nucleic acid molecule. Numerous amplification reactions have been reported in the art, including a polymerase chain reaction (PCR) (U.S. Pat. Nos. 4,683,195, 4,683,202, and 4,800,159), a reverse transcription-polymerase chain reaction (RT-PCR) (Sambrook et al., Molecular Cloning. A Laboratory Manual, 3rd ed. Cold Spring Harbor Press (2001)), methods by Miller, H. I. (WO 89/06700) and Davey, C. et al. (EP 329,822), a ligase chain reaction (LCR) (17, 18), Gap-LCR (WO 90/01069), a repair chain reaction (EP 439,182), transcription-mediated amplification (TMA) (19) (WO 88/10315), self-sustained sequence replication (20) (WO 90/06995), selective amplification of target polynucleotide sequences (U.S. Pat. No. 6,410,276), a consensus sequence primed polymerase chain reaction (CP-PCR) (U.S. Pat. No. 4,437,975), an arbitrarily primed polymerase chain reaction (AP-PCR) (U.S. Pat. Nos. 5,413,909 and 5,861,245), nucleic acid sequence based amplification (NASBA) (U.S. Pat. Nos. 5,130,238, 409,818, 5,554,517, and 6,063,603), loop-mediated isothermal amplification (LAMP) (23), and loop mediated isothermal amplification (LAMP) (23), but are not limited thereto. Other usable amplification methods are disclosed in U.S. Pat. Nos. 5,242,794, 5,494,810, and 4,988,617.

According to another preferable embodiment of the present invention, the composition is used on a biological sample isolated from a suspected prostate cancer patient.

As used herein, the term “biological sample” refers to a sample used to detect microRNA to verify the occurrence of prostate cancer. Preferably, the biological sample includes urine, blood, serum, plasma, tissues, or cells, but is not limited thereto.

The microRNA molecule of the present invention shows accurate diagnostic performance even in cases of using urine supernatants not containing cell-derived materials, among various biological samples, leading to a very simple and convenient diagnostic procedure.

In accordance with another aspect of the present invention, there is provided a kit for the diagnosis of prostate cancer, including the composition.

According to a preferable embodiment of the present invention, the kit includes a microarray chip including the probe having a sequence complementary to the nucleotide sequence of hsv1-miR-H18 or hsv2-miR-H9-5p of the present invention.

In the microarray chip of the present invention, the probe is used as a hybridizable array element, and is immobilized on a substrate. A preferable substrate is a suitable solid or semi-solid substrate, such as a membrane, filter, chip, slide, wafer, fiber, magnetic or nonmagnetic bead, gel, tubing, plate, polymer, microparticle, and capillary tube. The hybridizable array elements are arranged and immobilized on the substrate. Such immobilization is conducted by a chemical binding method or a covalent binding method, such as using UV light. For example, the hybridizable array elements may be bound to a glass surface that is modified to contain an epoxy compound or an aldehyde group, or may be bound to a polylysine-coated surface by using UV light. Further, the hybridizable array elements may be bound to a substrate via a linker (e.g., an ethylene glycol oligomer and a diamine).

In accordance with still another aspect of the present invention, there is provided a biomarker for the diagnosis of prostate cancer, including microRNA hsv1-miR-H18 or hsv2-miR-H9-5p.

In accordance with still another aspect of the present invention, there is provided a method for determining the expression level of microRNA as a biomarker of prostate cancer, to provide information necessary for the diagnosis of prostate cancer, the method including a step of determining the expression level of microRNA hsv1-miR-H18 or hsv2-miR-H9-5p in a biological sample.

According to a preferable embodiment, the biological sample is urine, blood, serum, plasma, tissues, or cells

In accordance with still another aspect of the present invention, there is provided a method for the diagnosis of prostate cancer, the method including a step of determining the expression level of microRNA hsv1-miR-H18 or hsv2-miR-H9-5p.

According to a preferable embodiment of the present invention, the determining of the expression level is conducted by using a primer or probe having a sequence complementary to a nucleotide sequence of microRNA hsv1-miR-H18 or hsv2-miR-H9-5p.

According to another preferable embodiment of the present invention, the determining of the expression level is conducted by a gene amplification reaction using the primer.

According to still another preferable embodiment of the present invention, the determining of the expression level of the RNA hsv1-miR-H18 or hsv2-miR-H9-5p is conducted on a biological sample isolated from a suspected prostate cancer patient.

According to still another preferable embodiment of the present invention, the biological sample is urine, blood, serum, plasma, tissues, or cells.

### Advantageous Effects

Features and advantages of the present invention are summarized as follows.

(i) The present invention provides a novel use of microRNA hsv1-miR-H18 or hsv2-miR-H9-5p for the diagnosis of prostate cancer.

(ii) According to hsv1-miR-H18 and hsv2-miR-H9-5p of the present invention, accurate diagnosis can be attained on even a patient within the PSA gray zone, in which accurate diagnosis is not attained by the PSA assay.

(iii) The present invention can be applied to various biological samples, and shows accurate diagnostic performance even in cases of using urine supernatants excluding cell-derived materials, leading to a simple and convenient diagnostic procedure.

(iv) According to the present invention, the diagnosis of prostate cancer can be carried out accurately, promptly, and at low costs.

### BRIEF DESCRIPTION OF THE DRAWINGS

FIG. 1 illustrates an overview of the study design in association with the present invention.

FIG. 2 illustrates clinical characteristics of patients and controls in training set, test set-1, test set-2, biopsy set, and matched set.

FIG. 3 illustrates box plots showing expression levels of urinary miRNAs in BPH controls (n=117) and CaP patients (n=180). Significantly high expression levels of hsa-miR-615-3p, ebv-miR-BART4, has-miR-4316, hsv1-miR-H18, and hsv2-miR-H9-5p were showed in urine samples from CaP patients (P<0.05 for all miRNAs).

FIG. 4 illustrates diagnostic performance of urinary miRNAs hsv1-miR-H18 and hsv2-miR-H9-5p compared with serum PSA levels. FIG. 4A shows receiver operating characteristic (ROC) curves for all patients (n=180) and controls (n=117); FIG. 4B shows ROC curves for patients within the PSA gray zone (≦3 ng/ml and ≧0 ng/ml); and FIG. 4C shows ROC curves for biopsy cases (n=102). The optimal cutoff points were determined based on the ROC curve analysis that yielded the highest combined sensitivity and specificity for detection.

FIG. 5 illustrates expression patterns of five candidate miRNAs in matched prostate tissue, urine, and serum samples.

FIG. 6 illustrates correlation between hsv1-miR-H18 and hsv2-miR-H9-5p levels in matched tissue, urine, and serum samples. Black circles indicate prostate cancer patients and open circles indicate BPH controls. There was a significant correlation between hsv1-miR-H18 (A) and hsv2-miR-H9-5p (B) levels in tissue and urine samples (Spearman correlation=0.369, P<0.001, and Spearman correlation=0.552, P<0.001, respectively). On the other hand, there was only a weak correlation between the levels in tissue and serum samples.

FIG. 7 illustrates stability and reproducibility of urine cell-free miRNAs after normalization to the total RNA concentration. Four urine samples were obtained from two healthy volunteers. First morning voided urine samples, and urine samples after 1, 2, and 4 hours were obtained. There was no significant difference in the miRNA level in the any experiment sample.

FIG. 8 illustrates direct staining results of benign prostatic hyperplasia (BPH) tissue, normal (non-cancer) tissue, and cancer tissue, using QD565-hsv1-miR-H18/hsv2-miR-H9 beacon-specific detection system. In panels A-C, the fluorescence reactivity of two miRNAs was higher in the prostate cancer (CaP) tissue (panel C) and the normal tissue (panel B) surrounded by cancer tissue rather than in the benign prostatic hyperplasia (BPH) tissue (panel A). Panels D-E show expression level of hsv1-miR-H18 (panel D) or hsv2-miR-H9 (panel E) in the prostatic tissue measured by MB imaging.

### MODE FOR CARRYING OUT THE INVENTION

Hereinafter, the present invention will be described in detail with reference to examples. These examples are only for illustrating the present invention more specifically, and it will be apparent to those skilled in the art that the scope of the present invention is not limited by these examples.

### Examples

### Methods

1. Cases and Controls

Samples from 402 prostate cancer (CaP) patients, 246 benign prostatic hyperplasia (BPH) patients, and 102 patients who underwent prostate biopsy were used in the study. Patients who underwent radical prostatectomy or palliative transurethral resection of the prostate (TURP) with histologically confirmed primary adenocarcinoma were included. Controls were selected from a database of BPH patients who underwent TURP. Biopsy cases were selected prospectively in consecutive patients who underwent transrectal ultrasound-guided biopsy. In CaP and BPH patients, first morning voided urine was collected prior to surgery. As for the biopsy patients, spot urine samples were obtained immediately before the procedure. The collected urine samples were centrifuged at 25,000 rpm for 15 minutes, and the supernatant was divided into 1 ml of aliquots, which were then stored at −20° C. until use. Similarly, serum samples were obtained on the morning of the operation and stored at −20° C. until sample preparation. All prostate tissues were macro-dissected, typically within 15 minutes of surgical resection, and then all tissue specimens were pathologically examined. Gleason grades and TNM 2002 staging were used as prognostic factors.

2. Purification of MicroRNA (miRNA)

miRNAs were isolated from 500 μl of urine using the Genolution Urine miRNA Purification Kit (Genolution Pharmaceuticals Inc., Seoul, Korea), and from 200 μl of serum using the Genolution Serum miRNA Purification Kit (Genolution Pharmaceuticals Inc) according to the manufacturer's protocol. The miScript Reverse Transcription Kit (Qiagen Korea, Seoul, Korea) was used to reverse transcribe the miRNAs, according to the manufacturer's recommended protocol. A mixture of template RNA, 5× miScript buffer, miScript reverse transcriptase mix, and water with RNase removed) (total volume: 20 μl) was shortly centrifuged and incubated at 37° C. for 60 minutes. Then, the miScript reverse transcriptase mix was inactivated and left on ice.

3. miRNA Microarray for Urine and Data Analysis

Total RNA was extracted from urine samples using the RecoverAll Total Nucleic Acid Isolation Kit (Life Technologies, Carlsbad, Calif., USA) according to the manufacturer's recommended protocol. RNA quantity and integrity were examined with the RNA 6000 Pico Chip Kit (Agilent Technologies, Santa Clara, Calif., USA) and Agilent 2100 Bioanalyzer. Profiling of miRNA was performed using the Agilent Human miRNA Microarray Release 16.0 platform, which screened 1,205 human and 144 viral miRNAs.

4. miRNA Detection by Real-Time PCR

To quantify miRNA expression, real-time PCR amplification was performed using the Rotor-Gene Q (Qiagen, Valencia, Calif.) and miScript PCR Starter Kit (Qiagen Korea, Seoul, South Korea). Chemically synthesized RNA oligonucleotides (Cosmo Genetech, Seoul, Korea) corresponding to the target miRNAs were used to generate standard curves. The standard curves ranged from 30 to 3×10⁴ copies. Target miRNAs were amplified using forward primers listed in Table 1. Real-time PCR conditions followed the manufacturer's recommended protocol, and all samples were run in triplicate.

TABLE 1

| miRNAs | miRNA ID | Primer |
|---|---|---|
| Candidate | hsa-miR-1273d | gaacccatgaggttg |
| miRNA | | aggctgcagt |
| | | (SEQ ID NO: 1) |
| | hsa-miR-3650 | aggtgtgtctgtaga |
| | | gtcc |
| | | (SEQ ID NO: 2) |
| | hsa-miR-429 | taatactgtctggta |
| | | aaaccgt |
| | | (SEQ ID NO: 3) |
| | hsa-miR-99a-5p | aacccgtagatccga |
| | | tcttgtg |
| | | (SEQ ID NO: 4) |
| | hsa-miR-815-3p | ccgagcctgggtctc |
| | | cctctt |
| | | (SEQ ID NO: 5) |
| | ebv-miR-BART4 | gacctgatgctgctg |
| | | gtgtgct |
| | | (SEQ ID NO: 6) |
| | hsv1-miR-H18 | cccgcccgccggacg |
| | | ccgggacc |
| | | (SEQ ID NO: 7) |
| | hsv2-miR-H9-5p | ctcggaggtggagtc |
| | | gcggt |
| | | (SEQ ID NO: 8) |
| | hsa-miR-1323 | tcaaaactgaggggc |
| | | attttct |
| | | (SEQ ID NO: 9) |
| | hsa-miR-4316 | ggtgaggctagctgg |
| | | tg |
| | | (SEQ ID NO: 10) |
| Reference | hsa-miR-518a | ctgcaaagggaagcc |
| miRNA | | ctttc |
| | | (SEQ ID NO: 11) |
| | hsa-miR-3605 | tgaggatggatagca |
| | | aggaagcc |
| | | (SEQ ID NO: 12) |
| | hsa-miR-16 | tagcagcacgtaaat |
| | | attggcg |
| | | (SEQ ID NO: 13) |
| | RUN6-2 | agttcccctgcataa |
| | | ggatg |
| | | (SEQ ID NO: 14) |

5. Quantification of miRNA Concentration Using RiboGreen

There was no reference urinary miRNA that has been recognized until now and thus the miRNA expression of all samples was normalized to the total RNA concentration in urine. The Quant-iT™ RiboGreen RNA and Kit (Invitrogen, Grand Island, N.Y.) were used to measure the total miRNA concentration purified from the samples.

6. Bio-Imaging Using QD₅₆₅ Nanoparticle-Conjugated Hsv1-miR-H18/Hsv2-miR-H9 Reactive Molecular Beacons

QD₅₆₅-hsv1-miR-H18/hsv2-miR-H9 beacons capable of specifically detecting hsv1-miR-H18 or hsv2-miR-H9 were manufactured by a conventional method (Yoon T J, Yu K N, Kim E, et al. Small 2006; 2: 209-15). Specific sequences of hsv1-miR-H18 and hsv2-miR-H9 beacons and the scrambled-beacon were as follows:

(SEQ ID NO: 17)
5′-NH2-TTCGCTGTGGTCCCGGCGTCCGGCCGGG-CGGGCGGGACCAC
AGCG-BHQ2-3′;
(SEQ ID NO: 18)
5′-NH2-TCGCTGTTCCGCGACTCCACCTCCGTGGCGACAACCGTCGGA
ACAGCG-BHQ2-3′;
and
(SEQ ID NO: 19)
5′-NH2-TTCGCTGTGGTCCCGGCGTTTGCCCCCACAGCG-BHQ2-3′.

Carboxyl QD nanoparticles (Life Technologies inc, Eugene, Calif.) were manufactured by the method described in existing literature (So M K, Loening A M, Gambhir S S, Rao J. Nature protocols 2006; 1: 1160-4). In order to increase the coupling efficiency between amine and carboxyl groups, the carboxyl QD nanoparticles, with the addition of N-ethyl-N′-dimethylaminopropyl carbodiimide (EDC), were allowed to react at room temperature for 1 hour to be covalently bonded with hsv1-miR-H18/hsv2-miR-H9 beacons (10 pM).

For quantitative images, whole-mount molecular beacons technology was applied (He S, Krens S G, Zhan H, et al., The Journal of pathology 2011; 225: 19-28).

Quantum dot (QD)₅₆₅-hsv1-miR-H18/hsv2-miR-H9 beacons were used as follows: QD₅₆₅-hsv1-miR-H18, -hsv2-miR-H9, and -hsv-miR-HS beacon probes (1:1,000; Bioneer, Daejeon, Korea) were used for detection.

Fluorescent images were obtained at 405 nm and 543 nm using Zeiss LSM710 on Axiovert (Carl Zeiss, Oberkochen, Germany) having the excitation HeNe laser beam, and 4× or 20× objective lens was used. Confocal stack was treated using Zeiss ZEN2011 software for maximum intensity projection. To define virtual mask, the shape thereof was drawn using a Region of Interest (ROI) function of Zeiss ZEN 2011 software, and the unit is expressed as arbitrary fluorescence intensity (AFI). For 3D analysis, z-stack was loaded in the constructor module using no sub-sampling.

7. Statistical Analysis

Data visualization and analysis were performed on the training set using GeneSpring GX (ver. 7.3) software (Agilent Technologies, Santa Clara, Calif.). Differentially expressed miRNAs were identified using Student t-test with a P-value cut-off of 0.05 and a fold-change threshold of 2.0. One-way analysis of variance (ANOVA) was used to analyze the correlation between miRNA expression and clinicopathological characteristics.

For test set-2, data values were converted into natural log values to normalize highly sloping distributions of miRNAs, and then the results were analyzed through reverse conversion. However, for test set-1 and its matched set, Mann-Whitney U test was used to compare miRNA levels between respective test groups. Receiver operating characteristic (ROC) curves were used to evaluate the diagnostic performance of the candidate miRNAs, and the optimal cutoff points for candidate markers were determined based on the highest combined sensitivity and specificity for detection in the ROC curve analysis. Spearman correlation coefficient was used to evaluate the correlation between miRNA levels in the matched samples. The statistical analysis was performed using IBM SPSS Statistics ver. 21.0 (SPSS Inc., Chicago, Ill.), and a P-value of less than 0.05 was considered statistically significant.

### Results

1. Overview of Study Design

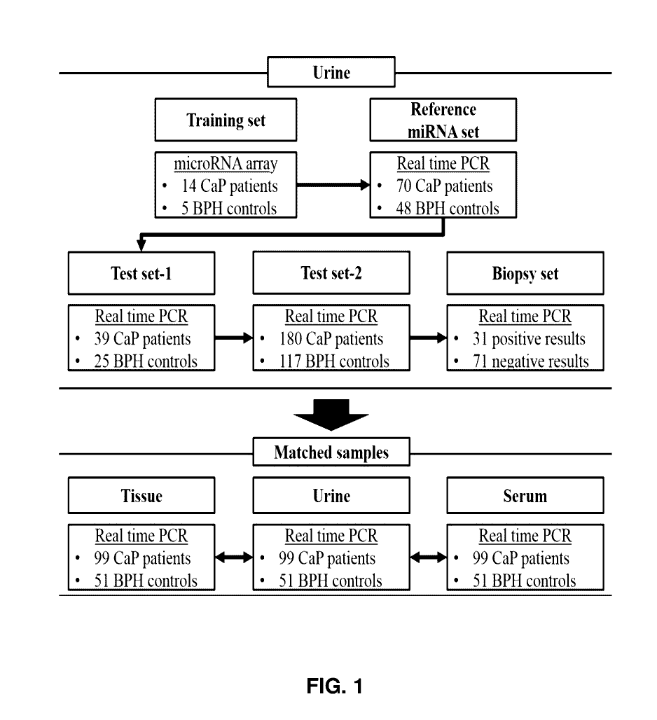

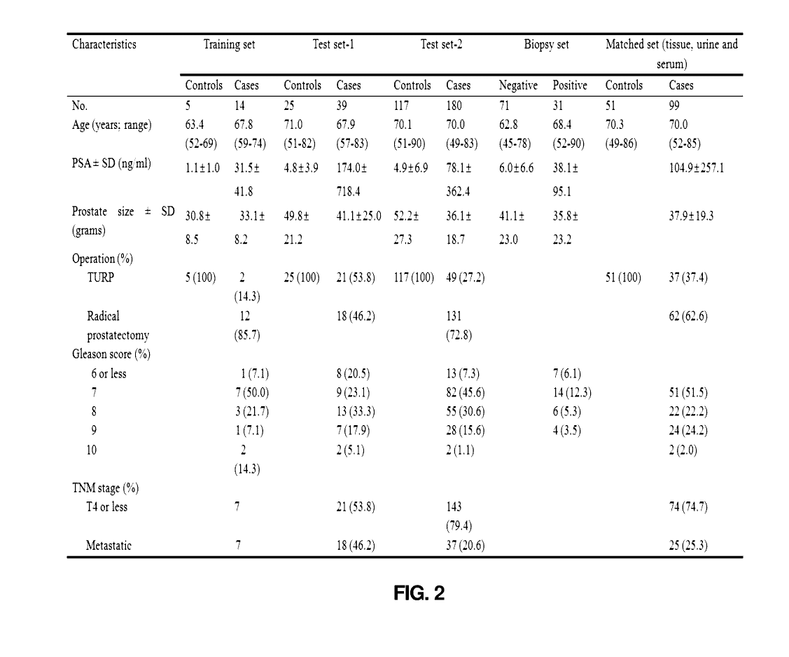

The training set including urine samples from 14 CaP patients (localized stage for seven cases and advanced stage for the other seven cases) and urine samples from five BPH controls was used to perform miRNA array analysis. Prior to the test of candidate miRNAs using real-time PCR, the urine samples from 70 Cap patients and 48 BPH controls were used to identify reference markers. Test set-1 (urine samples from 39 CaP patients and urine samples from 25 BPH controls), test set-2 (samples from 180 CaP patients and 117 BPH controls), and biopsy set (71 negative samples and 31 positive samples) were used to identify candidate urine miRNAs. Finally, matched serum, prostate tissue, and urine samples were obtained from 99 CaP patients and 51 BPH controls to investigate the correlation between levels of candidate miRNAs. FIG. 1 illustrates an overview of the study design in association with the present invention; and FIG. 2 illustrates clinical characteristics of patients and controls in training set, test set-1, test set-2, biopsy set, and matched set.

2. Selection of Candidate Urinary miRNAs from miRNA Array

Ten miRNAs were selected from miRNA array data of the training set (Table 2). It was validated through PCR analysis that the expression levels of hsa-miR-1273d, -3650, -429 and -99a-5p in the urine samples from the CaP patients were significantly lower than those from the BPH controls (P<0.05). In addition, the expression levels of hsa-miR-615-3p, ebv-miR-BART4, and hsv1-miR-H18 were significantly high in the urine samples from the CaP patients (P<0.05). In addition, the expression levels of hsv2-miR-H9-5p, and hsa-miR-1323 and -4316 were higher in the urine samples from the CaP patients at the advanced stage rather than in the urine samples from the patients at the localized stage of cancer.

In order to identify urinary reference markers, four candidate miRNAs (two markers from miRNA array [hsa-miR-518a and -3605] and two conventional markers [hsa-miR-16 and RUN6-2]) were evaluated by real-time PCR. However, none of the four candidate markers were suitable to be used as a urinary reference marker. Therefore, reference miRNA was not used in the present study. Instead, miRNA expression was normalized to the total RNA concentration.

TABLE 2

| | | BPH controls vs. | | Localized vs. | |
|---|---|---|---|---|---|
| | | CaP cases | | advanced stage | |
| | | | P | | P |
| No | miRNA ID | FC | value | FC | value |
| 1 | hsa-miR-1273d | 0.02 | 0.0000 | 20.98 | 0.0222 |
| 2 | hsa-miR-3650 | 0.35 | 0.0009 | 0.75 | 0.1465 |
| 3 | hsa-miR-429 | 0.09 | 0.0010 | 1.67 | 0.3761 |
| 4 | hsa-miR-99a-5p | 0.05 | 0.0000 | 11.92 | 0.0051 |
| 5 | hsa-miR-615-3p | 158.30 | 0.0001 | 818.51 | 0.0000 |
| 6 | ebv-miR-BART4 | 6.51 | 0.0387 | 1.34 | 0.6242 |
| 7 | hsv1-miR-H18 | 3.82 | 0.0000 | 1.89 | 0.0022 |
| 8 | hsv2-miR-H9-5p | 0.99 | 0.9594 | 2.74 | 0.0000 |
| 9 | hsa-miR-1323 | 1.16 | 0.7684 | 6.10 | 0.0012 |
| 10 | hsa-miR-4316 | 2.05 | 0.1857 | 5.76 | 0.0019 |

In Table 2, BPH control: benign prostatic hyperplasia control, CaP case: prostate cancer patient

3. Evaluation of Ten Candidate Urinary miRNAs from Test Set-1

Ten miRNAs from test set-1 were evaluated through real-time PCR (Table 3). The expression patterns of hsa-miR-1273d, -3650, and -615-3p, ebv-miR-BART4, hsv1-miR-H18, hsv2-miR-H9-5p, and hsa-miR-4316 were consistent with those observed in the training set. However, the expression patterns of hsa-miR-429, -99a-5p, and -1323 were different from those observed in the array data. The expression levels of ten miRNAs had no correlation with the clinical stage (non-metastatic vs. metastatic), Gleason score (≦7 vs.≧8), or PSA level (<20 ng/ml vs.≧20 ng/ml).

TABLE 3

| | | BPH controls (n = 25) | CaP patients (n = 39) | |
|---|---|---|---|---|
| | | (median (IQR; ×10² | (median (IQR; ×10² | |
| | | copies/miRNA | copies/miRNA | |
| No. | miRNA ID | concentration)) | concentration)) | P value |
| 1 | hsa-miR-1273d | 22133.1 (15288.1-32364.9) | 1951.6 (821.2-14307.3) | 0.010 |
| 2 | hsa-miR-3650 | 5403.6 (2906.9-6869.4) | 1482.7 (450.7-5376.9) | 0.025 |
| 3 | hsa-miR-429 | 10.8 (2.0-83.9) | 109.5 (2.7-257.6) | 0.208 |
| 4 | hsa-miR-99a-5p | 1429.7 (1267.1-3323.0) | 1743.8 (731.6-2428.0) | 0.615 |
| 5 | hsa-miR-615-3p | 761.1 (359.5-1532.8) | 3025.1 (1929.1-9686.2) | 0.027 |
| 6 | ebv-miR-BART4 | 7591.8 (939.0-25973.4) | 25403.4 (5085.7-60056.8) | <0.001 |
| 7 | hsv1-miR-H18 | 2228.4 (752.0-5650.6) | 11967.8 (2445.3-35353.9) | <0.001 |
| 8 | hsv2-miR-H9-5p | 832.8 (681.9-1872.1) | 2055.6 (726.0-6721.6) | 0.002 |
| 9 | hsa-miR-1323 | 1581.3 (356.7-6996.7) | 3140.5 (1762.5-10732.1) | 0.171 |
| 10 | hsa-miR-4316 | 3445.5 (808.2-10247.9) | 8307.2 (2130.5-16347.5) | 0.001 |

In Table 3, BPH control: benign prostatic hyperplasia control, CaP case: prostate cancer patient.

4. Evaluation of Seven Candidate Urinary miRNAs from Test Set-2

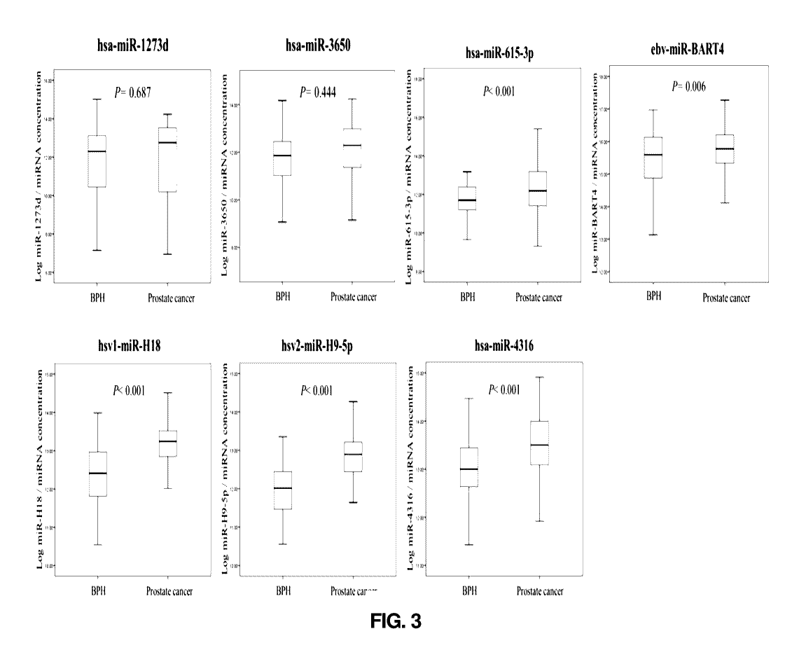

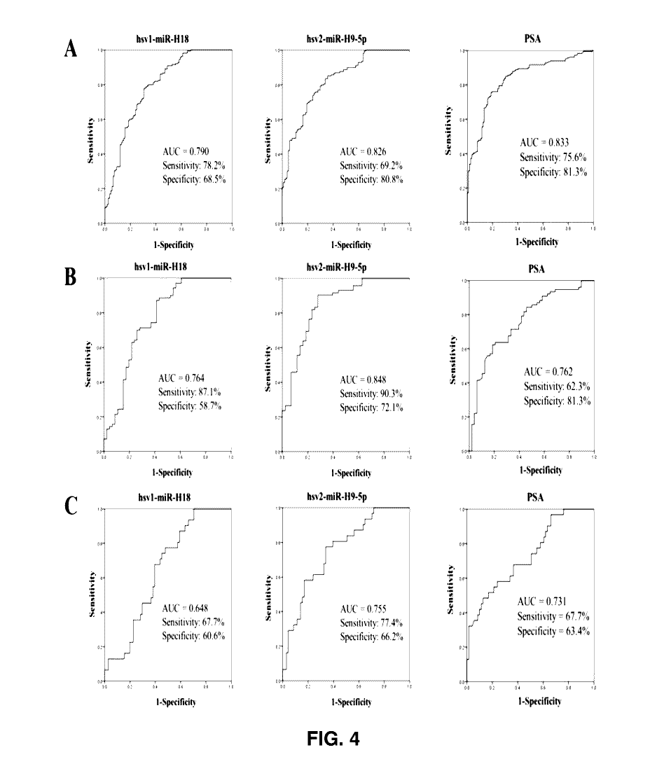

Among seven urinary miRNAs identified from test set-2, the expression levels of hsa-miR-615-3p, ebv-miR-BART4, hsv1-miR-H18, hsv2-miR-H9-5p, and hsa-miR-4316 showed a significant difference between BPH controls and CaP patients (P<0.05)(FIG. 3). Especially, the expression levels of hsv1-miR-H18 and hsv2-miR-H9-5p was higher in the CaP patients rather than BPH controls (P<0.001). ROC curve analysis was performed to determine how well the expression levels of hsv1-miR-H18 and hsv2-miR-H9-5p were differentiated between CaP patients and controls (FIGS. 4A and 4B). The level of hsv1-miR-H18 in the urine had sensitivity of 78.2% and specificity of 68.5%, and showed an area under the curve (AUC) value of 0.790, whereas the level of hsv2-miR-H9-5p in the urine had sensitivity of 69.2% and specificity of 80.8%, and showed an AUC value of 0.826. For patients within the PSA gray zone (PSA≧3 ng/ml and ≦10 ng/ml), the AUC values for hsv1-miR-H18 and hsv2-miR-H9-5p were 0.761 and 0.848, respectively, and thus, these miRNAs showed better diagnostic performance compared with the measurement of serum PSA level (AUC=0.762). In addition, the expression levels of hsa-miR-1273 and -4316, and hsv1-miR-H18 were higher in CaP patients with Gleason score of ≧8 rather than patients with Gleason score of ≦7 (P=0.050, P=0.027, and P=0.036, respectively). However, there were no significant differences in expression levels of seven urinary miRNAs between different cancer stages or different PSA levels.

5. Evaluation of Two Candidate Urinary miRNAs from Biopsy Set

The diagnostic performance of urinary hsv1-miR-H18 and hsv2-miR-H9-5p was re-evaluated from patients underwent transrectal prostate biopsy. AUC values for hsv1-miR-H18 and hsv2-miR-H9-5p were 0.648 and 0.755, respectively. Therefore, hsv2-miR-H9-5p showed discrimination ability, comparable to that of serum PSA levels (AUC-0.731)(FIG. 4C).

6. Correlations of Five Candidate miRNAs Between Matched Tissue, Serum, and Urine Samples

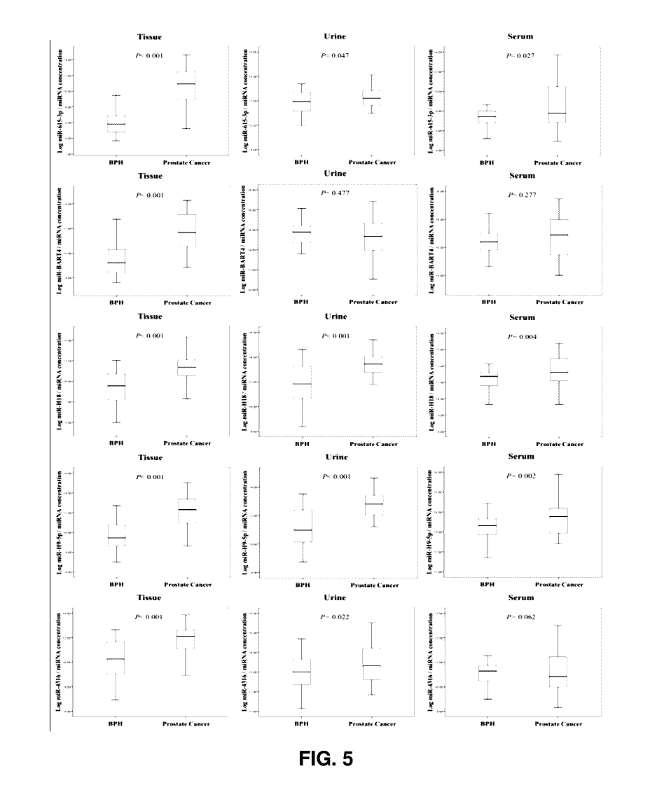

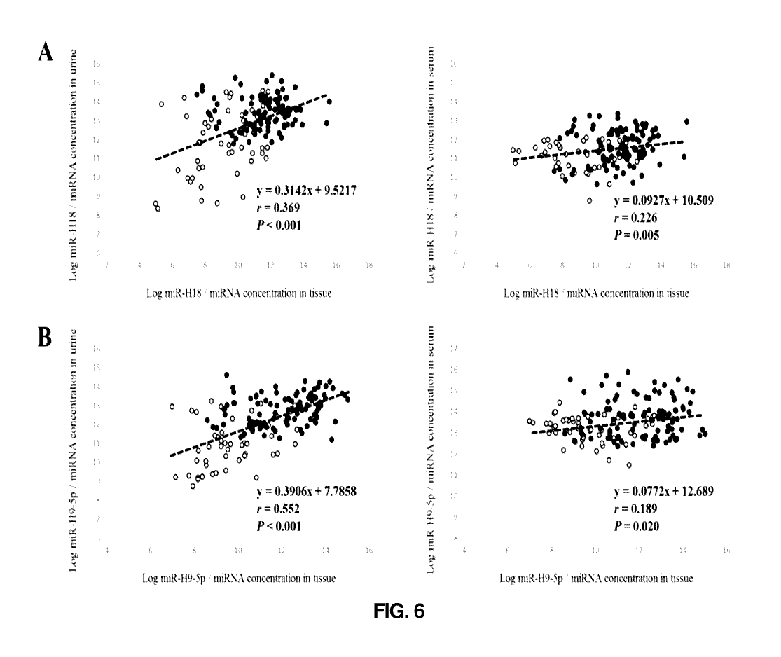

Last, the expression levels of five representative miRNAs were measured in matched tissue, serum, and urine samples from 99 CaP patients and 51 BPH controls. As a result, hsa-miR-615-3p, hsv1-miR-H18, hsv2-miR-H9-5p, and hsa-miR-4316 showed similar expression patterns in tissue and urine samples, whereas hsv1-miR-H18 and hsv2-miR-H9-5p showed a correlation in tissue and serum samples (FIG. 5). Most importantly, only hsv1-miR-H18 and hsv2-miR-H9-5p expression levels were similarly increased in tissue, urine, and serum samples (FIG. 6).

7. Supplementary Study Results

(1) Identification of Candidate Urinary Reference miRNAs

Urinary reference miRNA candidates were selected from miRNA microarrays derived from 14 CaP patients (localized stage for seven cases and advanced stage for the other seven cases) and 5 BPH controls. The present inventors selected hsa-miR-518a and -3605, which were detectable in all the urine samples from 21 patients and expressed at similar levels in CaP and BPH patients. In addition, the present inventors included hsa-miR-16 and RUN6-2, which were widely used as reference miRNA or reference small RNA in body fluids or tissues. The four candidate reference miRNAs were validated by real-time PCR in 70 urine samples from CaP patients and 48 samples from BPH controls. The expression level between patients and controls were compared, and the expression stability was analyzed using the NormFinder program. NormFinder is a Microsoft Excel add-in program that uses an ANOVA-based model to calculate stability values from different subgroups within a panel of candidate genes by combining intra- and intergroup expression variation (10).

Table 4 shows stability and expression levels of the candidate markers. The expression of the four candidate miRNAs was significantly different between samples from CaP patients and BPH patients (P<0.05). The NormFinder stability values were too high for use as urinary reference markers. Therefore, they could not be used in the present study. Instead, miRNA expression was normalized to the total RNA concentration measured using RiboGreen.

TABLE 4

| | | BPH controls (n = 48) | CaP cases (n = 70) | |
|---|---|---|---|---|
| | | (median (IQR; ×10² | (median (IQR; ×10² | |
| | Stability | copies/miRNA | copies/miRNA | P- |
| miRNA ID | value* | concentration)) | concentration)) | value |
| hsa-miR-518a | 408991.5 | 4098.6 (3566.3-10639.7) | 2670.6 (1904.3-4097.3) | <0.001 |
| hsa-miR-3605 | 647977.5 | 8842.4 (2952.6-44122.7) | 2781.7 (1015.4-21381.8) | 0.033 |
| hsa-miR-16 | 119927.7 | 1030.7 (525.7-2048.6) | 619.2 (320.0-1018.6) | 0.040 |
| RUN6-2 | 61037.2 | 752.8 (302.1-2537.1) | 230.5 (92.6-585.5) | <0.001 |

*High expression stability is indicated by a low stability value.

In Table 4, BPH control: benign prostatic hyperplasia control, CaP case: prostate cancer patient

(2) Stability and Reproducibility of Urinary Candidate miRNAs Normalized to Total miRNA Concentration

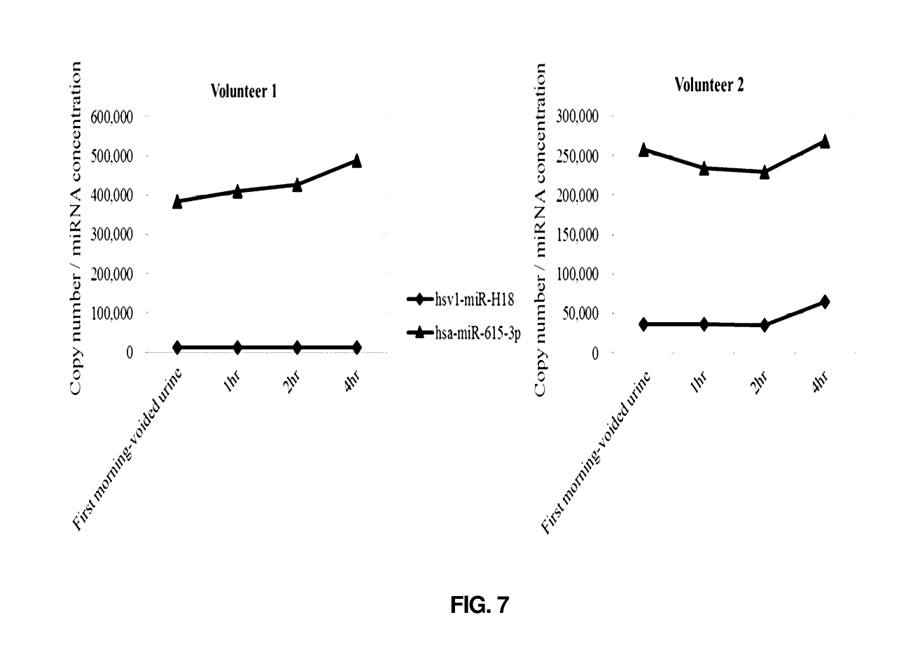

To examine stability and reproducibility of miRNAs in urine samples with different collection times, first morning voided urine was obtained from healthy volunteers. Then, samples were further collected after 1, 2, and 4 hours. There were no differences in hsa-miR-615-3p and hsv1-miR-H18 levels in all study samples (see FIG. 7). This means that the candidate urinary miRNAs normalized to the total miRNA concentration had stability and reproducibility regardless of the time point of collection.

8. Quantification of Hsv1-miR-H18 and Hsv2-miR-H9-5p Expression Levels in Prostate Cancer Tissue and Benign Prostatic Hyperplasia Control Tissue Through Molecular Beacon-Based Direct Staining Analysis

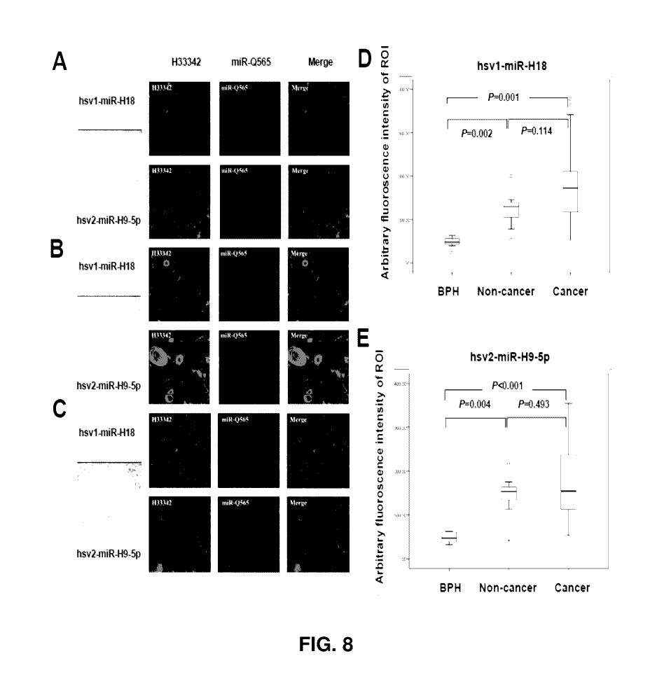

To bio-image and quantify hsv1-miR-H18 and hsv2-miR-H9-5p in tissue samples, molecular beacon-based imaging analysis, which is a nonradioactive method of detecting particular sequences of nucleic acids, was performed. Quantum dot (QD) bundle nanoparticles QD₅₆₅-hsv1-miR-H18/hsv2-miR-H9 were manufactured as a molecular beacon probe, which is an internally quenched fluorophore, and then used to directly stain hsv1-miR-H18/hsv2-miR-H9 in the prostate tissue of benign prostatic hyperplasia (BPH) (panel A of FIG. 8), normal tissue surrounded by cancer (panel B of FIG. 8), or the prostate cancer (CaP) tissue (panel C of FIG. 8). The fluorescence reactivity of hsv1-miR-H18/hsv2-miR-H9 was higher in the prostate cancer (CaP) tissue and the normal tissue surrounded by cancer tissue rather than benign prostatic hyperplasia (BPH) tissue. These results were consistent with RT-PCR-based study results (panels D and E of FIG. 8).

### Discussion

The above study results established that virus-encoded miRNAs (hsv1-miR-H18 and hsv2-miR-H9-5p) in urine can be used as a diagnostic biomarker for CaP, and revealed that the expression patterns of these miRNAs were similar between the prostate tissue and serum. The study results of the present invention first reported the correlation between the virus-encoded miRNAs and CaP. Pfeffer et al. first identified EBV-encoded pre-miRNAs in B95-8 cells in 2004 (10). Since then, more than 250 virus-encoded miRNAs have been identified. The roles and functions thereof have not yet been understood, but virus miRNAs can target viruses and human cell transcripts (11). Virus-encoded miRNAs are grouped into two classes: analogues of host miRNAs and virus-specific miRNAs. Virus-encoded miRNAs mimic specific host miRNAs and negatively regulate transcription via the same target docking sites as their counterpart host miRNAs (12, 13). However, some of virus-encoded miRNAs truly mimic host miRNAs (11). Most of virus-encoded miRNAs are virus-specific and are thought to regulate host cells in several ways: (1) by regulation of the latent-lytic switch; (2) by supporting viral replication through promoting cell survival, proliferation, and/or differentiation; and (3) by modulating immune responses (11). Viruses such as herpes virus, Epstein-Barr virus, and Marek's disease virus (these viruses are all associated with tumorigenesis), encode miRNAs. However, tumorigenesis is not the primary aim of these viruses, and it is an unfortunate consequence of their ability to alter the cell cycle, prevent cell death, and avoid immune responses (14). So far, there was no study result on the establishment of the correlation between virus-encoded miRNAs and CaP. There were evidences that CaP is associated with infection and inflammation caused by bacteria and viruses, and thus more studies should be carried out, but it is possible that hsv1-miR-H18 and hsv2-miR-H9-5p perform a key role in tumors. The extraction and quantification of urinary miRNAs is very tough work since urinary miRNAs exist in a very low concentration. Actually, clinical tests will be difficult if a large quantity of urine was needed to extract miRNAs. Here, the present inventors extracted RNAs (miRNAs and small RNAs) from 500 μl of urine. The use of such a small quantity of sample precludes the calculation of RNA concentration using a spectrometer. In addition, the RNA concentration in the urine was highly variable and dependent on a hydrated state, kidney function, and time for collection. Therefore, a reliable marker was needed to normalize the variation between samples. So far, human miRNA, such as miR-16 or RNU6B, has been used as an endogenous control (15, 16). However, several studies revealed that miR-16 is a diagnostic or prognostic biomarker for CaP (17-19), miR-16 is no longer considered to be a reliable reference miRNA. The present inventors evaluated whether four miRNAs, that is, hsa-miR-518a, hsa-miR-3605, hsa-miR-16, and RUN6-2, were suitable to be used as miRNA (see supplement study results). However, no miRNA was validated to be suitable, and thus the present inventors focused to the normalization of miRNA concentration to the total RNA concentration. The RiboGreen RNA quantification kit had very high sensitivity, and allowed the measurement of accurate RNA concentration (1-50 ng/ml). When the miRNA levels were normalized to miR-16 and the diagnostic performance thereof were examined in test set-2, the diagnostic performance was not satisfactory. Shen, et al. examined serum from CaP patients using a method similar to the method described herein (6). They generated standard curves using chemically synthesized RNA oligonucleotides, and then calculated copy number (per μl) of candidate miRNAs using the standard curves. However, they did not determine RNA levels in serum, which means that it could not be guaranteed that respective samples contain the same quantity of RNA. Therefore, the present inventors are convinced that miRNA levels should be normalized to the total RNA (miRNA and small RNA) concentration in various body fluids. This measurement method has possibility to introduce an innovation in miRNA studies in body fluids. A number of studies report promising results regarding the use of putative miRNAs in blood or urine as a diagnostic or prognostic marker for CaP, but the method of the present invention has the following advantages, compared with the above study results.

First, the present inventors experimented on 402 cases and 246 controls, and validated on five different cohorts. Such a study has the biggest scale in view of the examination of putative miRNAs in the body fluid from CaP patients. The present inventors found that cell-free hsv1-miR-H18 and hsv2-miR-H9-5p levels were consistently high in urine samples from three different cohorts. In addition, expression patterns in matched prostate tissue, urine, and serum samples were shown to be the same. These miRNAs showed excellent discrimination ability between CaP and BPH (control). Further, hsv2-miR-H9-5p had detection ability, comparable to that of serum PSA levels in patients underwent transrectal prostate biopsy, and this fact suggests that the miRNAs can actually be a useful diagnostic marker for CaP.

Second, the present inventors determined the levels of cell-free miRNA in 500 μl of urine supernatants rather than in urine pellets containing cellular materials, such as erythrocytes, leukocytes, and even cancer cells. The non-uniform cellular components in the pellets may influence the reliability of miRNAs as biomarkers. So far, there have been two study results regarding urinary miRNAs as biomarkers for CaP (7, 20). Bryant et al. suggested that miR-107 and miR-574-3p are present at higher levels in the urine of CaP patients than in that of healthy controls (7). However, since the urine samples in the above study were collected after transrectal digital massage, the samples were very highly variable even though they contain a lot of information about prostate. In addition, Srivastava et al. reported that miR-205 and miR-214 are downregulated in the urine of CaP patients, and thus can be used to discriminate CaP patients from healthy controls. They selected miRNAs from a tissue miRNA array, and examined the levels thereof in urine samples using RNU48 for normalization, which is a different approach from that used in our study. The present inventors evaluated the diagnostic performance by selecting candidate miRNAs from the urine-based array and then normalizing the levels thereof to the total RNA level in urine, tissue, and serum samples. Since there is no validated reference miRNA at the present time, the method of the present invention is thought to be the most ideal approach.

Third, it is interesting that virus-encoded hsv1-miR-H18 and hsv2-miR-H9-5p showed better diagnostic performance than PSA levels in the patients within the PSA gray zone. There is no cutoff value recognized for PSA in the detection of CaP, and thus numerous diagnostic markers were developed and tested in an attempt to increase diagnostic performance. However, these markers show poor reproducibility and are too complex to use in the clinical setting. The method of the present invention can easily and accurately normalize miRNA levels to the total RNA level using the RiboGreen RNA kit and employing only 500 μl of urine. In addition, the marker of the present invention is obtained from the urine sample, and thus can reflect well prostate conditions. In cases where the matched samples were examined, more significant correlation and more excellent AUC values for tissues were shown in the urine sample rather than the serum sample. Therefore, the present inventors believe that the miRNA of the present invention is very useful as a diagnostic marker for CaP, and, especially, will be very useful when used in combination with PSA measurement. Finally, conventional studies validated miRNAs in serum or urine using healthy persons as controls, but in the method of the present invention, the experiments were conducted by setting BPH patients underwent TURP as controls. Therefore, the mean PSA for test set-2 was 4.9 ng/ml. In addition, BPH patients had bigger prostates than normal groups. The present inventors consider that these controls represent well actual clinical situations. In these conditions, hsv1-miR-H18 and hsv2-miR-H9-5p showed better discrimination ability.

### CONCLUSION

The present results first established that virus-encoded miRNAs are associated with CaP. These results indicate that urinary cell-free miRNAs can be useful in the diagnosis of CaP. Especially, these results indicate that encoded miRNA hsv1-miR-H18 and hsv2-miR-H9-5p can be very important urinary diagnostic markers for CaP. These two miRNAs show better diagnostic performance even on the patients within the PSA gray zone.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### REFERENCES

1. Ambros V. Nature. Sep. 16 2004; 431(7006):350-355.
2. Bartel D P. Cell. Jan. 23 2004; 116(2):281-297.
3. Calin G A, Sevignani C, Dumitru C D, et al. Proc Natl Acad Sci USA. Mar. 2 2004; 101(9):2999-3004.
4. Lu J, Getz G, Miska E A, et al. Nature. Jun. 9 2005; 435(7043):834-838.
5. Chen C Z. N Engl J Med. Oct. 27 2005; 353(17):1768-1771.
6. Shen J, Hruby G W, McKiernan J M, et al. The Prostate. Sep. 15 2012; 72(13):1469-1477.
7. Bryant R J, Pawlowski T, Catto J W, et al. British journal of cancer. Feb. 14 2012; 106(4):768-774.
8. Delgado P O, Alves B C, Gehrke Fde S, et al. Tumour biology: the Journal of the International Society for Oncodevelopmental Biology and Medicine. April 2013; 34(2):983-986.
9. Bland J M, Altman D G. Bmj. Apr. 27 1996; 312(7038):1079.
10. Pfeffer S, Zavolan M, Grasser F A, et al. Science. Apr. 30 2004; 304(5671):734-736.
11. Skalsky R L, Cullen B R. Annual review of microbiology. 2010; 64: 123-141.
12. Grundhoff A, Sullivan C S. Virology. Mar. 15 2011; 411(2):325-343.
13. Kincaid R P, Burke J M, Sullivan C S. Proceedings of the National Academy of Sciences of the United States of America. Feb. 21 2012; 109(8): 3077-3082.
14. Zhao Y, Xu H, Yao Y, et al. PLoS pathogens. February 2011; 7(2):e1001305.
15. Chang K H, Mestdagh P, Vandesompele J, Kerin M J, Miller N. BMC cancer. 2010; 10:173.
16. Schaefer A, Jung M, Miller K, et al. Experimental and molecular medicine. Nov. 30 2010; 42(11):749-758.
17. Watahiki A, Wang Y, Morris J, et al. PloS one. 2011; 6(9):e24950.
18. Leite K R, Tomiyama A, Reis S T, et al. Urologic oncology. August 2013; 31 (6):796-801.
19. Mahn R, Heukamp L C, Rogenhofer S, von Ruecker A, Muller S C, Ellinger J. Urology. May 2011; 77(5):1265 e1269-1216.
20. Srivastava A, Goldberger H, Dimtchev A, et al. PloS one. 2013; 8(10):e76994.

### INDUSTRIAL APPLICABILITY

MicroRNAs of the present inventions, hsv1-miR-H18 or hsv2-miR-H9-5p, can be used for a method and kit for the diagnosis of prostate cancer.

1. A composition for the diagnosis of prostate cancer, containing an agent capable of detecting microRNA hsv1-miR-H18 or hsv2-miR-H9-5p.

2. The composition of claim 1, wherein the agent is a primer or probe having a sequence complementary to a nucleotide sequence of microRNA hsv1-miR-H18 or hsv2-miR-H9-5p.

3. The composition of claim 2, wherein the primer is used for a gene amplification reaction.

4. The composition of claim 1, wherein the composition is used on a biological sample isolated from a suspected prostate cancer patient.

5. The composition of claim 4, wherein the biological sample is urine, blood, serum, plasma, tissues, or cells.

6. A kit for the diagnosis of prostate cancer, comprising the composition of claim 1.

7. The kit of claim 6, wherein the kit is a microarray chip.

8. A biomarker for the diagnosis of prostate cancer, comprising microRNA hsv1-miR-H18 or hsv2-miR-H9-5p.

9. A method for determining the expression level of microRNA as a biomarker of prostate cancer, to provide information necessary for the diagnosis of prostate cancer, the method comprising a step of determining the expression level of microRNA hsv1-miR-H18 or hsv2-miR-H9-5p in a biological sample.

10. The method of claim 9, wherein the biological sample is urine, blood, serum, plasma, tissues, or cells.

11. A method for the diagnosis of prostate cancer, the method comprising a step of determining the expression level of microRNA hsv1-miR-H18 or hsv2-miR-H9-5p.

12. The method of claim 11, wherein the determining of the expression level is conducted by using a primer or probe having a sequence complementary to a nucleotide sequence of microRNA hsv1-miR-H18 or hsv2-miR-H9-5p.

13. The method of claim 11, wherein the determining of the expression level is conducted by a gene amplification reaction using the primer.

14. The method of claim 11, wherein the determining of the expression level of microRNA hsv1-miR-H18 or hsv2-miR-H9-5p is conducted on a biological sample isolated from a suspected prostate cancer patient.

15. The method of claim 14, wherein the biological sample is urine, blood, serum, plasma, tissues, or cells.


SEQUENCE LISTING (19 sequences)

SEQ ID NO: 1 - DNA, 25 bp, Artificial Sequence
  Synthetic (PCR primer)
gaacccatgaggttgaggctgcagt

SEQ ID NO: 2 - DNA, 19 bp, Artificial Sequence
  Synthetic (PCR primer)
aggtgtgtctgtagagtcc

SEQ ID NO: 3 - DNA, 22 bp, Artificial Sequence
  Synthetic (PCR primer)
taatactgtctggtaaaaccgt

SEQ ID NO: 4 - DNA, 22 bp, Artificial Sequence
  Synthetic (PCR primer)
aacccgtagatccgatcttgtg

SEQ ID NO: 5 - DNA, 21 bp, Artificial Sequence
  Synthetic (PCR primer)
ccgagcctgggtctccctctt

SEQ ID NO: 6 - DNA, 22 bp, Artificial Sequence
  Synthetic (PCR primer)
gacctgatgctgctggtgtgct

SEQ ID NO: 7 - DNA, 23 bp, Artificial Sequence
  Synthetic (PCR primer)
cccgcccgccggacgccgggacc

SEQ ID NO: 8 - DNA, 20 bp, Artificial Sequence
  Synthetic (PCR primer)
ctcggaggtggagtcgcggt

SEQ ID NO: 9 - DNA, 22 bp, Artificial Sequence
  Synthetic (PCR primer)
tcaaaactgaggggcattttct

SEQ ID NO: 10 - DNA, 17 bp, Artificial Sequence
  Synthetic (PCR primer)
ggtgaggctagctggtg

SEQ ID NO: 11 - DNA, 20 bp, Artificial Sequence
  Synthetic (PCR primer)
ctgcaaagggaagccctttc

SEQ ID NO: 12 - DNA, 23 bp, Artificial Sequence
  Synthetic (PCR primer)
tgaggatggatagcaaggaagcc

SEQ ID NO: 13 - DNA, 22 bp, Artificial Sequence
  Synthetic (PCR primer)
tagcagcacgtaaatattggcg

SEQ ID NO: 14 - DNA, 20 bp, Artificial Sequence
  Synthetic (PCR primer)
agttcccctgcataaggatg

SEQ ID NO: 15 - RNA, 23 nt, Artificial Sequence
  Synthetic (mirco RNA hsv1-miR-H18)
cccgcccgccggacgccgggacc

SEQ ID NO: 16 - RNA, 20 nt, Artificial Sequence
  Synthetic (micro RNA hsv2-miR-H9-5p)
cucggagguggagucgcggu

SEQ ID NO: 17 - DNA, 45 bp, Artificial Sequence
  Synthetic (Molecular beacon sequence for hsv1-miR-H18)
ttcgctgtggtcccggcgtccggccgggcgggcgggaccacagcg

SEQ ID NO: 18 - DNA, 48 bp, Artificial Sequence
  Synthetic (Molecular beacon sequence for hsv2-miR-H9)
tcgctgttccgcgactccacctccgtggcgacaaccgtcggaacagcg

SEQ ID NO: 19 - DNA, 33 bp, Artificial Sequence
  Synthetic (Sequence for scrambled-beacon)
ttcgctgtggtcccggcgtttgcccccacagcg